(19) United States
(12) Patent Application Publication
(10) Pub. No.: US 2009/0093871 A1
(43) Pub. Date: Apr. 9, 2009

(54) Medical Implant With Internal Drug Delivery System

(75) Inventors: Susan Rea, Santa Rosa, CA (US); Feridun Ozdil, Santa Rosa, CA (US); Robert Rajalingam, Santa Rosa, CA (US); Noreen F. Moloney, Galway (IE); Jeffrey Allen, Santa Rosa, CA (US); Matthew J. Birdsall, Santa Rosa, CA (US); Darrel Untereker, Oak Grove, MN (US); Jeffrey Gross, Vancouver (CA); Mark Dolan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/868,742

(22) Filed: Oct. 8, 2007

(51) Int. Cl. A61F 2/06

(52) U.S. Cl. 623/1.11; 604/523; 623/1.42

(57) ABSTRACT

A system for treating a vascular condition includes a catheter and a stent disposed on the catheter. The stent includes tubing having a wall defining a central lumen and a plurality of holes. The system further includes a therapeutic agent disposed within the central lumen of the tubing. A method of manufacturing a therapeutic agent carrying stent includes inserting a therapeutic agent within a therapeutic agent delivery system into the central lumen of a hollow metal tube and forming a stent framework from the hollow tube.

### TECHNICAL FIELD

This invention relates generally to biomedical devices that are used for treating vascular conditions. More specifically, the invention relates to a therapeutic agent eluting stent formed from hollow tubing having a therapeutic agent delivery system disposed within the central lumen of the hollow tubing.

### BACKGROUND OF THE INVENTION

Stents are generally cylindrical-shaped devices that are radially expandable to hold open a segment of a vessel or other anatomical lumen after implantation into the body lumen.

Various types of stents are in use, including expandable and self-expanding stents. Expandable stents generally are conveyed to the area to be treated on balloon catheters or other expandable devices. For insertion into the body, the stent is positioned in a compressed configuration on the delivery device. For example, the stent may be crimped onto a balloon that is folded or otherwise wrapped about the distal portion of a catheter body that is part of the delivery device. After the stent is positioned across the lesion, it is expanded by the delivery device, causing the diameter of the stent to expand. For a self-expanding stent, commonly a sheath covering the stent is retracted, allowing the unconstrained stent to expand.

Stents are used in conjunction with balloon catheters in a variety of medical therapeutic applications, including intravascular angioplasty to treat a lesion such as plaque or thrombus. For example, a balloon catheter device is inflated during percutaneous transluminal coronary angioplasty (PTCA) to dilate a stenotic blood vessel. When inflated, the pressurized balloon exerts a compressive force on the lesion, thereby increasing the inner diameter of the affected vessel. The increased interior vessel diameter facilitates improved blood flow. Soon after the procedure, however, a significant proportion of treated vessels restenose.

To reduce restenosis, stents, constructed of metals or polymers, are implanted within the vessel to maintain lumen size. The stent is sufficiently longitudinally flexible so that it can be transported through the cardiovascular system. In addition, the stent requires sufficient radial strength to enable it to act as a scaffold and support the lumen wall in a circular, open configuration. Configurations of stents include a helical coil, and a cylindrical sleeve defined by a mesh, which may be supported by a stent framework of struts or a series of rings fastened together by linear connecter portions.

Stent insertion may cause undesirable reactions such as inflammation resulting from a foreign body reaction, infection, thrombosis, and proliferation of cell growth that occludes the blood vessel. Stents capable of delivering one or more therapeutic agents have been used to treat the damaged vessel and reduce the incidence of deleterious conditions including thrombosis and restenosis.

Polymer coatings applied to the surface of the stents have been used to deliver drugs or other therapeutic agents at the placement site of the stent. The coating is sometimes damaged during expansion of the stent at the delivery site, causing the coating to chip off the stent and release flakes of the polymer coating, which reduces the effective dose of the drug at the treatment site, and under some circumstances, may result in emboli in the microvasculature. Also, some polymers have been found to be irritating to the surrounding tissues during long term implantation.

It would be desirable, to provide an implantable stent having a delivery system for one or more therapeutic agents disposed within a hollow stent framework. Polymers comprising the delivery system would be prevented from contacting tissues and the delivery system would be protected from damage during delivery to the treatment site. Such a stent would overcome many of the limitations and disadvantages of the stents described above.

### SUMMARY OF THE INVENTION

One aspect of the present invention provides a system for treating a vascular condition. The system includes a catheter and a stent disposed on the catheter. The stent comprises a tubing including a wall defining a central lumen and a plurality of holes. The system further includes a therapeutic agent disposed within the central lumen of the tubing.

Another aspect of the invention provides a stent. The stent comprises a stent framework including a tubing. The tubing includes a wall defining a central lumen and a plurality of holes. A therapeutic agent delivery system is disposed within the central lumen of the stent framework. The stent also includes at least one therapeutic agent carried by the therapeutic agent delivery system, wherein the therapeutic agent delivery system controls an elution rate of the therapeutic agent.

Another aspect of the invention provides a method for manufacturing a therapeutic agent carrying stent. The method includes loading at least one therapeutic agent within a delivery system. The method further comprises inserting the delivery system into the central lumen of a hollow porous metal tube, and forming a stent framework from the hollow tube.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic illustration of a system for treating a vascular condition including a therapeutic agent carrying stent coupled to a catheter, in accordance with one embodiment of the present invention;

FIG. 2A is a schematic illustration of a portion of a hollow tubing having holes evenly distributed throughout the tubing wall, in accordance with the present invention;

FIG. 2B is a schematic illustration of a portion of porous hollow tubing having holes of different sizes on each lateral side of the tubing wall in accordance with the present invention;

FIG. 2C is a schematic illustration of a portion of hollow tubing having pores clustered laterally on one side of the tubing wall, in accordance with the present invention;

FIG. 2D is a schematic illustration of a portion of hollow tubing having holes clustered longitudinally along the length of the tubing wall, in accordance with the present invention;

FIG. 3A is a schematic illustration of a portion of a stent framework formed from hollow tubing in a contracted configuration, in accordance with the present invention;

FIG. 3B is a schematic illustration of a portion of a stent framework formed from hollow tubing in an expanded configuration, in accordance with the present invention;

FIG. 4A is a schematic illustration of a cross section of a stent comprised of hollow tubing and having holes through the tubing wall that forms the lumen of the stent and cavities in the wall of the tubing that forms the ablumenal surface, in accordance with the present invention;

FIG. 4B is a schematic illustration of a cross section of a hollow tubing having holes through the tubing wall and cavities in the wall, in accordance with the present invention;

FIG. 5A is a schematic illustration of a contracted stent formed from the stent framework shown in FIG. 3A and 3B, in accordance with the present invention;

FIG. 5B is a schematic illustration of an expanded stent formed from the stent framework shown in FIG. 3A and 3B, in accordance with the present invention; and

FIG. 6 is a flow diagram of one embodiment of a method for manufacturing a therapeutic agent carrying stent having a therapeutic agent delivery system in the internal lumen of the stent framework, in accordance with the present invention.

### DETAILED DESCRIPTION

Throughout this specification, like numbers refer to like structures.

The present invention is directed to a system for treating abnormalities of the cardiovascular system comprising a catheter and a therapeutic agent-carrying stent disposed on the catheter. In one embodiment, the stent comprises a hollow tubing having a therapeutic agent within the lumen of the tubing.

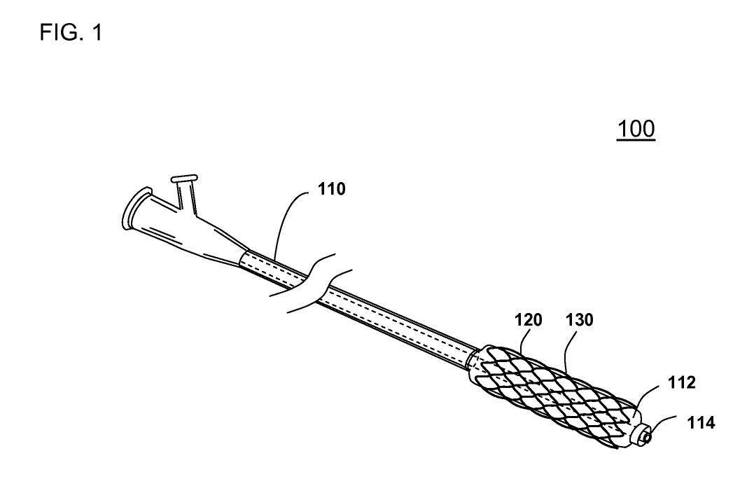

FIG. 1 illustrates one embodiment of a system 100 for treating a vascular condition. System 100 comprises therapeutic agent carrying stent 120 coupled to catheter 110. In an exemplary embodiment, catheter 110 includes a balloon 112 that expands and deploys therapeutic agent carrying stent 120 within a vessel of the body. After positioning therapeutic agent carrying stent 120 within the vessel with the assistance of a guide wire traversing through guide wire lumen 114 inside catheter 110, balloon 112 is inflated by pressurizing a fluid such as a contrast fluid or saline solution that fills balloon 112 via an inflation tube inside catheter 110. Therapeutic agent carrying stent 120 is expanded until a desired diameter is reached; then the inflation fluid is depressurized or pumped out, separating balloon 112 from therapeutic agent carrying stent 120 and leaving the therapeutic agent carrying stent 120 deployed in the vessel of the body. Alternately, catheter 110 may include a sheath that retracts to allow expansion of a self-expanding version of therapeutic agent carrying stent 120. Therapeutic agent carrying stent 120 includes a stent framework 130. In one embodiment of the invention, a porous zone is formed at the surface of at least a portion of metallic stent framework 130.

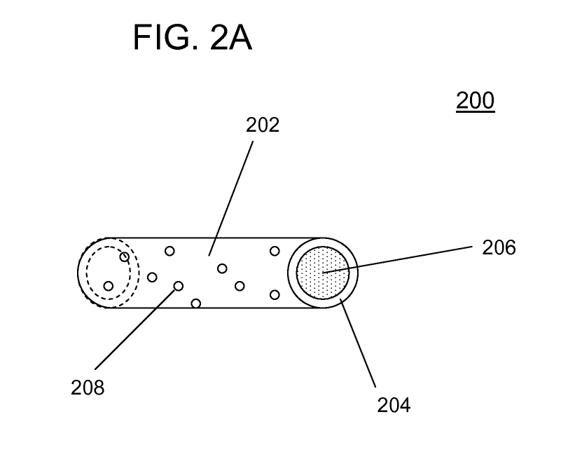

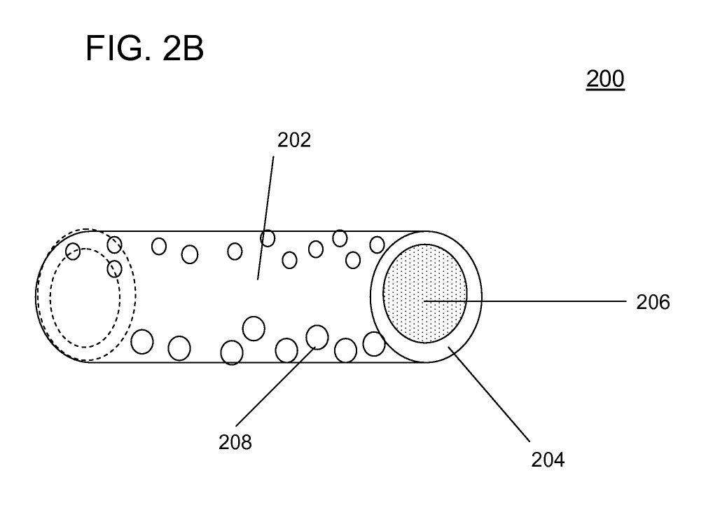

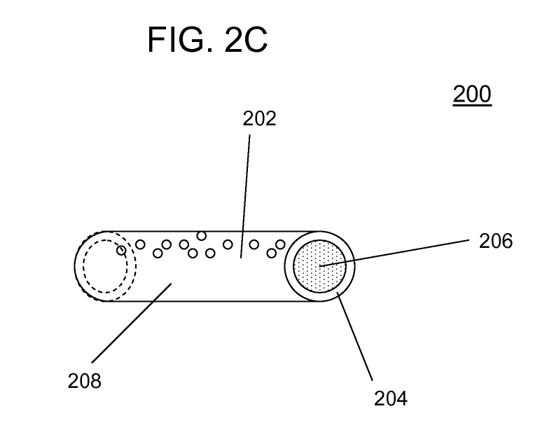

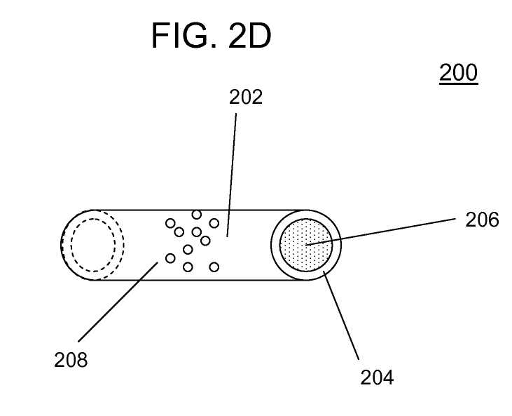

In one embodiment of the invention, stent 120 comprises hollow tube 200 as shown in FIG. 2A. Hollow tube 200 comprises a wall 204 defining a tube lumen 206. Wall 204 further defines a plurality of holes 208 that extend through the wall.

Wall 204 of hollow tube 200 comprises one or more of a variety of biocompatible metals such as stainless steel, titanium, magnesium, chromium, cobalt, nickel, gold, iron, iridium, chromium/titanium alloys, chromium/nickel alloys, chromium/cobalt alloys, such as MP35N and L605, cobalt/titanium alloys, nickel/titanium alloys, such as nitinol, platinum, and platinum-tungsten alloys. In another embodiment, wall 204 comprises one or more biocompatible thermoplastic polymers such as polyethylene, polypropylene, polymethyl methacrylate, polycarbonate, polyesters, polyamides, polyurethanes, polytetrafluoroethylene (PTFE), polyvinyl alcohol, polyether-amide elastomers, or any other suitable polymers.

Holes 208 extend through wall 204 and provide fluid communication between internal lumen 206 and exterior surface 202 of wall 204. Holes 208 may be of uniform size or variable in size. In one embodiment, holes 208 are distributed evenly throughout wall 204 as shown on FIG. 2A. In another embodiment, holes 208 are distributed evenly, but are of different sizes on each lateral side of wall 204 as shown in FIG. 2B. In another embodiment, holes 208 are clustered laterally on one side of stent wall 204 as shown in FIG. 2C, or clustered longitudinally in portions of stent wall 204 as shown in FIG. 2D. Holes 208 are formed in wall 204 by laser drilling, micromachining or any other appropriate method.

Wall 204 defines interior lumen 206 running longitudinally through the center of hollow tubing 200. In one embodiment of the invention, lumen 206 is filled with one or more therapeutic agents. In one embodiment, the therapeutic agent(s) is contained within a therapeutic agent delivery system. Various therapeutic agents, such as anticoagulants, antiinflammatories, fibrinolytics, antiproliferatives, antibiotics, therapeutic proteins or peptides, recombinant DNA products, or other bioactive agents, diagnostic agents, radioactive isotopes, or radiopaque substances may be used depending on the anticipated needs of the targeted patient population. The formulation containing the therapeutic agent may additionally contain excipients including solvents or other solubilizers, stabilizers, suspending agents, antioxidants, and preservatives, as needed to deliver an effective dose of the therapeutic agent to the treatment site. In one embodiment of the invention, the delivery system includes one or more polymers that provide a support matrix capable of being loaded with the therapeutic agents to be delivered, and releasing the therapeutic agents at an optimal rate. In one embodiment, the therapeutic agent delivery system is loaded into interior lumen 206 using a catheter, a syringe or a similar device. In one embodiment, the catheter has a tapered distal tip that connects to hollow tubing 200 and allows the therapeutic agent delivery system to be injected into lumen 206 of hollow tubing 200 under controlled pressure, determined in part by the diameter of the lumen of the catheter and the configuration of the tapered portion.

In one embodiment, two or more therapeutic agents are sequentially loaded into lumen 206 of hollow tubing 200. First, one therapeutic agent is loaded into the distal portion of lumen 206; then a polymer solution is injected that forms a biostable solid, or semisolid partition within lumen 206, effectively dividing lumen into separate chambers. Finally a second therapeutic agent is loaded into the proximal portion of lumen 206. Using this configuration, each therapeutic agent is delivered independently of the other therapeutic agent.

In one embodiment, the therapeutic agent delivery system includes polymeric microspheres or nanospheres. In this embodiment, the therapeutic agent is contained within the microspheres or nanospheres that release the therapeutic agent in a desired, predetermined elution profile. Polymeric microspheres or nanospheres may be biodegradable, biostable, or comprise a mixture of polymeric materials that are both biostable and biodegradable. Biodegradable polymers appropriate for the microspheres of the invention include polylactic acid, polyglycolic acid, and their copolymers, caproic acid, polyethylene glycol, polyanhydrides, polyacetates, polycaprolactones, poly(orthoesters), polyamides, polyurethanes and other suitable polymers. Biostable polymers appropriate for the microspheres of the invention include polyethylene, polypropylene, polymethyl methacrylate, polyesters, polyamides, polyurethanes, polytetrafluoroethylene (PTFE), polyvinyl alcohol, and other suitable polymers. These polymers may be used alone or in various combinations to give the microspheres unique properties such as controlled rates of degradation, or to form biostable microspheres with a biodegradable or bioerodable coatings on the surface of the microspheres.

In one embodiment, the therapeutic agent delivery system is a polymeric therapeutic agent delivery system. In this embodiment, the polymeric therapeutic agent delivery system is formulated as a liquid and injected into lumen 206 of hollow tube 200 under pressure. Once inside tubing 200, the polymeric delivery system thickens, or solidifies by cross linking of the polymers to form a polymer mesh-like matrix or hydrogel. Consequently, lumen 206 is filled with a viscous or solid material that, in addition to releasing the therapeutic agent, gives strength to tubing 200 and prevents kinking of tubing 200 when subjected to strain. Because the polymeric system forms a cross-linked mesh, the polymers remain within the lumen of hollow tubing 200 as the therapeutic agent is released through holes 208, providing controlled release of the therapeutic agent with no direct contact between the polymeric delivery system and the surrounding bodily tissues.

In one embodiment, polymers that do not cross-link are used to provide a support matrix capable of being loaded with the therapeutic agents to be delivered, and releasing the therapeutic agents at an optimal rate. In this embodiment, the size of holes 208 is selected so that the therapeutic agent molecules pass through holes 208, but the polymer molecules cannot, and therefore remain trapped within lumen 206. For example, the anticoagulants such as low molecular weight heparin or coumarin, having molecular weights of less than 5000 daltons may be delivered from a polymer matrix of polyethylene, polypropylene, polymethyl methacrylate, polyvinyl alcohol, or other suitable polymers having a molecular weight of greater than 20,000 daltons. In this case, the anticoagulant would pass freely through holes 208, while the polymer molecules would remain trapped within lumen 206.

In one embodiment, the polymer matrix binds to one or more therapeutic agents within interior lumen 206. One or more therapeutic agents partition into the cavities or, alternatively, are loaded into the cavities after the polymer matrix forms. Therapeutic agent release will depend on the partition coefficient of each therapeutic agent and the distance (diffusion path) to wall 204. In another embodiment, the polymer matrix is biodegradable and dissolves and washes out of interior lumen 206, thereby releasing each therapeutic agent. In this embodiment, the rate of dissolution and wash out of the polymer matrix regulates the rate of release of the therapeutic agent.

In one embodiment of the invention, the therapeutic agent delivery system comprises an aqueous insoluble oil, stable oil-in-water microemulsion or wax. Depending on its solubility, the therapeutic agent is dissolved or suspended within the non-aqueous, lipophilic delivery system. In this embodiment, the therapeutic agent molecules pass through holes 208 into the aqueous environment of the body, but the highly lipophilic components of the delivery system remain inside lumen 206. The rate of release of the therapeutic agent will depend on the surface area provided by holes 208.

In one embodiment of the invention, therapeutic agents differing in molecular diameter are delivered at different rates, depending on the size of holes 208. For example, the size of holes 208 may be selected according to the invention, so that a drug having a molecular weight that is less than 1000 and a peptide having a molecular weight of several thousand can each be delivered at optimal delivery rates. If needed, holes 208 may be of two sizes, one to accommodate each therapeutic agent. In this case, the release rate for each therapeutic agent will depend on the density of holes 208 of the appropriate size. In another embodiment, illustrated in FIG. 2B, holes 208 of one size are located on one lateral side of wall 204, and holes 208 of a second size are located on the contralateral side of wall 204, so that delivery of each therapeutic agent occurs only from one side of wall 204, thus facilitating directional delivery of the therapeutic agent.

In another embodiment, a therapeutic agent may be partially conjugated or derivatized with a polymer such as polyethylene glycol to increase the molecular size of some molecules, but not others. In this case the release rate of the therapeutic agent would be proportional to the ratio of derivatized to underivatized molecules.

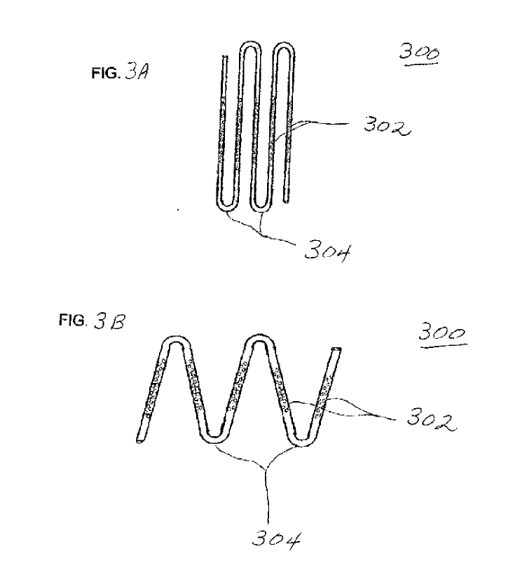

In one embodiment, stent wall 300 is formed by shaping filled tubing 200 into a zigzag configuration as shown in FIGS. 3A and 3B. In some embodiments, metallic stent framework 300 is formed into a tubular shape about a mandrel. FIG. 3A is a schematic illustration of a portion of stent framework 300 in a compressed configuration, as for example when it is mounted on a catheter during delivery. The stent has a reduced diameter and stent framework 300 is in a compressed configuration, with crown portions 304 acutely bent, and struts 302 approximately parallel to each other. When the stent is deployed at the treatment site, stent framework 300 is expanded. As the diameter of stent framework 300 increases, struts 302 move laterally away from each other and the angle formed by crown portions 304 is enlarged as shown in FIG. 3B. The opening and closing of the angle formed by crown portions 304 causes significant strain on crown portions 304. In contrast, little strain is placed on strut portions 302 of the stent. Strain is a measure of the displacement that can be applied to a material before the material breaks or tears. Strain is measured as the ratio of the change in length of the material to the original length of the material.

In one embodiment, filled tubing shown in FIG. 2C, with pores 208 clustered longitudinally, is shaped so that holes 208 are positioned in strut portions 302. Crown portions 304 are formed from the portions of filled tubing 200 having no holes. This configuration provides crown portions 304 with sufficient strength to prevent crown portions 304 from kinking when subjected to strain during expansion and contraction of stent framework 300.

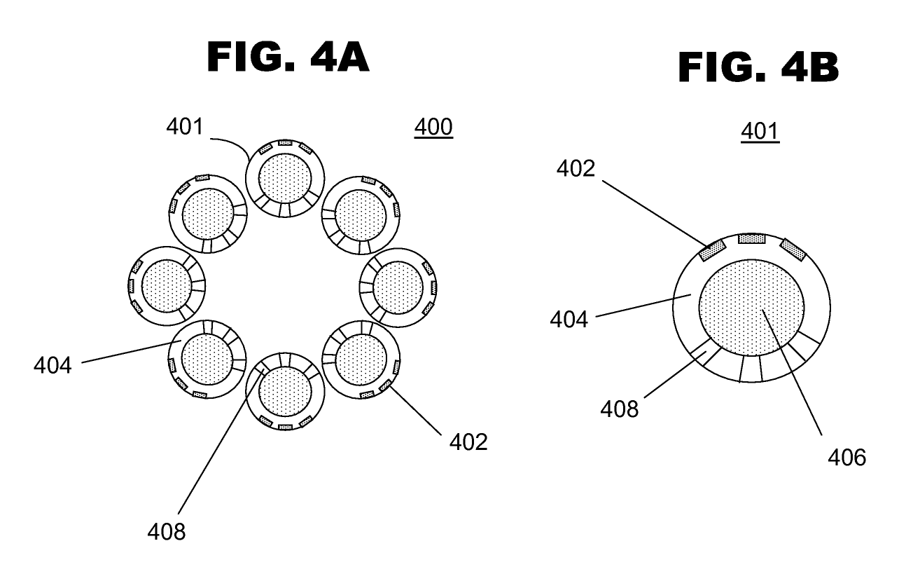

FIG. 4A is a schematic representation of a cross section of another embodiment of stent framework 400. In this embodiment, holes 408 are located on one lateral side of wall 404 as shown in FIG. 2C. On the contralateral side of wall 408 are pores or cavities 402. When stent framework 400 is shaped into a tubular configuration, holes 408 may be placed on the lumenal surface of stent framework 400, and cavities 402 on the ablumenal surface, as shown in FIG. 4A. Alternatively, holes 408 may be placed on the ablumenal surface of stent framework 400 and cavities 402 on the lumenal surface. In either case, two separate therapeutic agent delivery mechanisms are provided on stent framework 400.

FIG. 4B is a schematic representation of a cross section of the embodiment of hollow tubing 401 used to make stent framework 400 as shown in FIG. 4A. As in other embodiments, holes 408 penetrate wall 404 and provide fluid communication between tubular lumen 406 and the exterior of hollow tubing 401 allowing directional delivery of a therapeutic agent from lumen 406. Cavities 402 are formed in wall 404, but do not penetrate wall 404. Cavities 402 are of any appropriate diameter of uniform or variable size, and may range in size from nanopores to micropores. The cavities may be formed in wall 404 by laser drilling or micromachining. In one embodiment, a second therapeutic agent is placed in cavities 402 for directional delivery from the side of hollow tubing 401 that is contralateral to holes 408. In one embodiment, for example, an anticoagulant in an appropriate delivery system is loaded into inner lumen 406 and an antiproliferative agent is placed in cavities 402. A stent made from stent framework 400 having the configuration shown in FIG. 4A could then deliver the anticoagulant directionally into the blood stream from the lumenal surface of the stent and the antiproliferative agent directly to the vessel wall from the ablumenal surface of the stent.

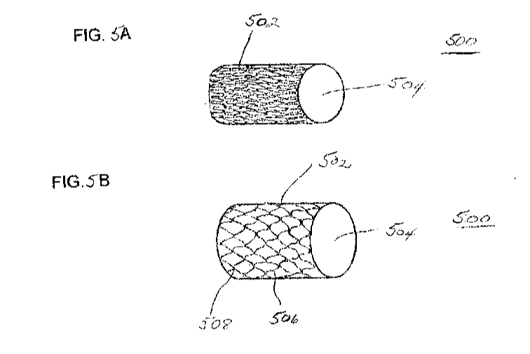

FIGS. 5A and 53B are schematic representations of stent 500 formed from stent framework 300 in a compressed and expanded configuration, respectively. To form stent 500, the flat planar configuration of stent framework 300 shown in FIGS. 3A and 3B is formed into the cylindrical or tubular structure shown in FIGS. 5A and 5B.

In one embodiment, filled hollow tubing 200, having holes 208 arranged as in FIG. 2C, is used to form stent 500. Holes 208 are placed on either exterior surface 502 or luminal surface 504 of stent 500, providing directional delivery of the therapeutic agent(s). In this embodiment, stent 500 having holes 208 on the external surface 502 can be placed at the treatment site and expanded against the wall of the blood vessel so that holes 208 are in direct contact with the vessel wall. An antiproliferative therapeutic agent, for example, can be delivered directly to the vessel wall from the internal lumen of the stent framework. In another embodiment, the stent framework is shaped so that holes 208 are on luminal surface 504 of finished stent 500. In this case a therapeutic agent, for example an anticoagulant can be delivered from the luminal surface of stent 500, where it is most effective. In either embodiment, a therapeutic agent can be delivered where it is most effective from a delivery system that is sequestered within the lumen of the stent framework and has no direct contact with tissues, thus providing an efficient delivery system while reducing the likelihood of adverse reactions to the polymers comprising the delivery system. In another embodiment, holes 208 of one diameter are on the luminal surface 504 of stent 500, and holes 208 having a different diameter are on the external surface of stent 500, allowing simultaneous delivery of one therapeutic agent, an anticoagulant, for example, from the luminal surface, and a second therapeutic agent such as an antiproliferative from the external surface, each at an optimal delivery rate.

In another embodiment, stent 500 is formed from a stent framework having holes 208 clustered longitudinally as shown in FIG. 2D, and FIGS. 3A and 3B. In this embodiment holes 208 are located on strut portions 506 of the stent framework. Crown portions 508, having a solid stent wall with no pores or holes to weaken them, are better able to withstand the strain placed on them during expansion and compression of the stent and provide strength to support the vessel wall.

In another embodiment, some or all of holes 208 of stent framework 300 are plugged with a sacrificial, biocompatible metal or metal alloy such as, for example, magnesium, silver, cobalt, copper, zinc, or iron. The material forming the plug may be applied to the holes by any means such as for example by spraying, dipping or any other means known in the art. The metal or metal alloy is selected that will erode in a clinically optimal time period. After placement of stent 500 in a blood vessel, the sacrificial metal will erode, opening holes 208 and allowing delivery of the therapeutic agent. This embodiment provides therapeutic agent delivery for an extended period of time after placement of the stent. In one embodiment, for example, an antiproliferative agent is loaded into lumen 206, and holes 208 are plugged with a thin layer of magnesium. Depending on the thickness of the layer, the magnesium is eroded in approximately two to four weeks after the stent is implanted in a blood vessel. In another embodiment, the magnesium layer plugging holes 208 erodes in about one day to about fourteen days. Holes 208 are then open and the antiproliferative agent is released from lumen 206.

In another embodiment, stent 500 is coated with a biocompatible, biodegradable polymer coating such as polylactic acid, polyglycolic acid, or their copolymers. Such a coating prevents loss of the therapeutic agent through the pores and or holes during handling and delivery of the stent. Once in place at the treatment site, the polymeric coating degrades and allows delivery of the therapeutic agent through holes 208. In another embodiment, the thickness of the polymeric layer is selected so that delivery of the therapeutic agent is extended over a period of time after placement of the stent.

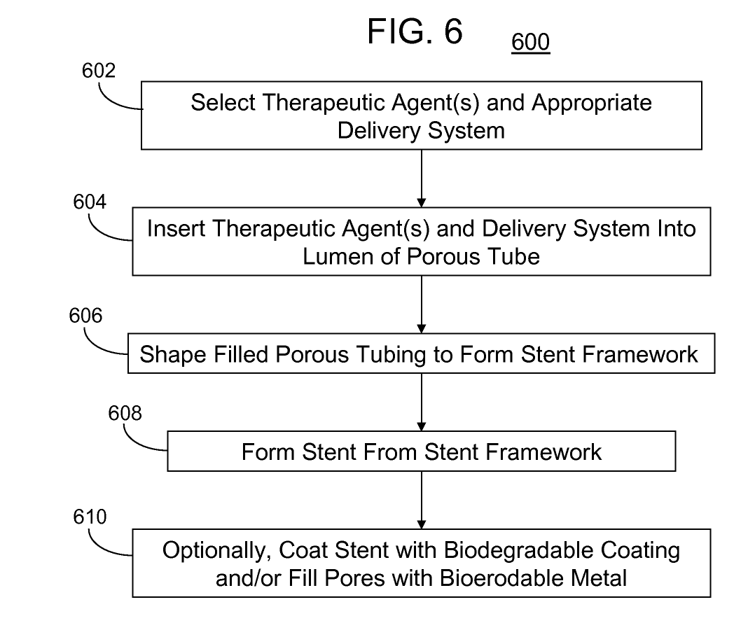

FIG. 6 is a flowchart of method 600 for manufacturing a therapeutic agent eluting stent in accordance with the present invention. The method includes selecting one or more therapeutic agents and an appropriate delivery system as indicated in Block 602. The therapeutic agents may include anticoagulants, antiinflammatories, fibrinolytics, antiproliferatives, antibiotics, radiopaque substances or other agents in a formulation containing the excipients needed to deliver an effective dose of the therapeutic agent to the treatment site. In some embodiments, the formulation will be a liquid or a suspension. In addition, in some embodiments, polymers that will control the rate of release of the therapeutic agent(s) will be included in the delivery system. In some embodiments, the polymers will have a sufficiently high Young's modulus to provide strength to the stent and prevent kinking of the stent framework in high strain regions.

Next, as indicated in Block 604, the therapeutic agent and delivery system are inserted into the lumen of a hollow porous hypotube. In various embodiments, the hypotube is metallic or polymeric and the holes and/or cavities are either randomly dispersed or clustered longitudinally or laterally. In one embodiment, the therapeutic agent and delivery system is formulated as a liquid and injected into the lumen of the porous tube. In one embodiment the delivery system forms a polymer mesh or hydrogel once inside the porous tube. In another embodiment, the delivery system includes polymers formulated as microspheres or nanospheres.

As indicated in Block 606, the filled porous tubing is shaped to form a stent framework. The stent framework provides the strength to support the vessel wall and a means of delivering one or more therapeutic agents at the treatment site.

Next, tubular stent 500 is formed from the stent framework 300, as indicated in Block 608. In one embodiment of the invention, holes 208 are clustered laterally on hollow tubing 200. Stent framework 500 can be oriented so that holes 208 are situated only on the external surface of stent 500, or on the luminal surface of stent 500 providing directional delivery of the therapeutic agent. In another embodiment, holes 208 are clustered on the strut portions 302 of stent framework 300. Finally, the manufacture of the stent is completed by, for example, placing a mesh over stent framework 300.

Optionally, as indicated in Block 610, a biodegradable polymer coating may be applied to the surface of stent framework 300 that will temporarily cover holes 208 and prevent loss of the therapeutic agent during handling and delivery of stent 500 to the treatment site. In one embodiment, holes 208 are filled with a sacrificial metal such as magnesium which will modify the rate of delivery of the therapeutic agent. After stent 500 is positioned at the treatment site, the sacrificial metal will erode and allow delivery of the therapeutic agent to take place.

The completed stent may then be compressed and mounted on a catheter, expanded at the delivery site, and otherwise handled as needed without chipping, flaking, or loss of the therapeutic agent. Once positioned at the treatment site the stent provides support for the vessel wall and therapeutic agent delivery without direct contact between tissues and the therapeutic agent delivery system.

While the invention has been described with reference to particular embodiments, it will be understood by one skilled in the art that variations and modifications may be made in form and detail without departing from the spirit and scope of the invention.

1. A system for treating a vascular condition comprising:
a catheter;
a stent disposed on the catheter, the stent comprising a tubing including a wall defining a central lumen, the wall further defining a plurality of holes through the wall; and
a therapeutic agent disposed within the central lumen of the tubing.

2. The system of claim 1 further comprising a therapeutic agent delivery system disposed within the central lumen.

3. The system of claim 1 further comprising a plurality of bioerodable plugs, the plurality of bioerodable plugs disposed within at least a portion of the plurality of holes.

4. The system of claim 3 wherein the erosion of at least one of the bioerodable plugs changes the rate of release of at least one therapeutic agent.

5. The system of claim 1 wherein the plurality of holes are sized to provide a predetermined rate of therapeutic agent delivery.

6. The system of claim 2 wherein the therapeutic agent delivery system comprises one or more polymers that control the rate of release of at least one therapeutic agent and provide radial strength to the stent framework.

7. The system of claim 6 wherein at least one of the polymers comprises microspheres or nanospheres.

8. The system of claim 6 wherein at least one of the polymers forms a porous matrix that includes cavities.

9. The system of claim 2 wherein the therapeutic agent delivery system comprises one or more aqueous insoluble substances selected from the group consisting of an oil, a stable oil-in-water microemulsion or a wax.

10. The system of claim 2 wherein the polymeric therapeutic agent delivery system is formulated as a liquid and injected under pressure into the central lumen of the stent framework.

11. The system of claim 1 further comprising a coating disposed on the surface of the stent framework.

12. The system of claim 1 wherein at least a portion of the stent framework is nonporous.

13. The system of claim 1 wherein the stent framework comprises one or more metals selected from the group consisting of magnesium, titanium, cobalt, chromium, cobalt/chromium alloys, nickel, platinum, iridium, gold, cobalt/titanium alloys, nickel/titanium alloys, platinum/tungsten alloys, chromium/nickel alloys, stainless steel, and other medically acceptable metals.

14. A stent comprising
a stent framework including a tubing, the tubing including a wall defining a central lumen, the wall further defining a plurality of holes through the wall;
a therapeutic agent delivery system disposed within the central lumen of the stent framework; and
at least one therapeutic agent carried by the therapeutic agent delivery system, wherein the therapeutic agent delivery system controls an elution rate of the therapeutic agent.

15. The stent of claim 14 wherein the stent framework comprises one or more metals selected from the group consisting of magnesium, aluminum titanium, cobalt, chromium, nickel, platinum, iridium, chromium/cobalt alloys, cobalt/titanium alloys, chromium/nickel alloys, stainless steel, and other medically acceptable metals.

16. The stent of claim 14 wherein the size of the holes in the tubing wall determines the rate of therapeutic agent delivery.

17. The stent of claim 14 wherein the location of the holes in the tubing wall determines the direction of therapeutic agent delivery.

18. The stent of claim 14 wherein the therapeutic agent delivery system includes one or more polymers that control the rate of release of at least one therapeutic agent and provide radial strength to the stent framework.

19. The stent of claim 18 wherein at least one of the polymers comprises microspheres or nanospheres.

20. The stent of claim 14 further comprising a coating disposed on the surface of the stent framework.

21. The stent of claim 14 wherein a portion of the stent framework is nonporous.

22. A method of manufacturing a therapeutic agent carrying stent, the method comprising:
loading at least one therapeutic agent within a therapeutic agent delivery system;
inserting the therapeutic agent delivery system within a central lumen of a hollow porous metal tube; and
forming a stent framework from the hollow tube.

23. The method of claim 22 wherein a portion of the metal tube is nonporous.

24. The method of claim 22 further comprising applying a coating to the exterior surface of the stent framework.

25. The method of claim 23 further comprising:
positioning one or more holes in the stent framework; and
directing therapeutic agent delivery to the target tissue via the one or more holes.

26. The method of claim 23 further comprising filling at least a portion of the holes in the stent framework with at least one of a bioabsorbable metal and metal alloy.

27. The method of claim 23 further comprising derivatizing at least a portion of the therapeutic agent molecules to obtain a desired molecular size.

28. The method of claim 23 further comprising attaching at least one therapeutic agent to a polymer comprising microspheres or nanospheres.
